(19) United States
(12) Patent Application Publication
(10) Pub. No.: US 2021/0228119 A1
(43) Pub. Date: Jul. 29, 2021

(54) MICRONEEDLE BIOSENSOR

(71) Applicant: KINDEVA DRUG DELIVERY L.P., St Paul, MN (US)

(72) Inventors: Chin-Yee NG, Oakdale, MN (US); Scott A. Burton, Woodbury, MN (US); Kandyce M. Bohannon, White Bear Lake, MN (US); Chu Yi, Taipei (TW)

(21) Appl. No.: 17/054,253

(22) PCT Filed: May 13, 2019
(86) PCT No.: PCT/IB2019/053947

§ 371 Date: Nov. 10, 2020

Related U.S. Application Data
(60) Provisional application No. 62/672,090, filed on May 16, 2018.

(51) Int. Cl. A61B 5/1486; A61B 5/00; A61B 5/145

(52) U.S. Cl. CPC A61B 5/14865; A61B 5/685; A61B 5/0031; A61B 5/6833; A61B 5/14546

(57) ABSTRACT

Device with probe located within hollow microneedles and having a ping to protect the probe from damage, and method of using the same to detect analyte.

### BACKGROUND

Use of transdermal devices, and particularly transdermal devices with microneedles, for detecting biological analytes is disclosed, for example, in PCT Publications WO2013/058879, WO2015/008018 and WO2013/58879; EP Patent No. 2898826, U.S. Pat. No. 8,560,059, and U.S. Patent Application Publication No. 2014/0275897.

WO2013/058879 discloses techniques, systems, and devices for biosensing, such as a device including an array of hollow needles, in which each needle includes a protruded needle structure including an exterior wall forming a hollow interior and an opening at a terminal end of the protruded needle structure exposing the hollow interior and a probe inside the exterior wall to interact with one or more chemical or biological substances that come into contact with the probe via the opening to produce a probe sensing signal and an array of wires that are coupled to the probes of the array of hollowed needles, respectively, each wire being electrically conductive to transmit the probe sensing signal produced by a respective probe.

### SUMMARY

A device comprises an array of one or more hollow needles that comprise a protruded needle structure including one or more exterior walls that define the sides of a hollow interior, a tip configured to face a biological surface that is exterior to the hollow microneedle, and a probe inside the exterior wall. The probe is located within the hollow interior defined by the one or more exterior walls, and is configured to interact with one or more chemical or biological substances that come into contact with the probe to produce a probe sensing signal. The device is characterized by a plug covering a tip of the hollow needle such that the plug is disposed between the probe and the exterior of the hollow microneedle, the plug being configured to at least partially dissolve in a biological environment to form an opening in the tip of the hollow interior and expose the probe to the biological environment. A method of detecting an analyte with the device is can include piercing a biological substrate with the hollow microneedles of the device and allowing the analyte to come into operative proximity with the probe to produce a probe signal that correlates to the presence of the analyte.

### BRIEF DESCRIPTION OF THE DRAWINGS

FIGS. 1A and 1B are views of a microneedle before and after dissolution of the plug;

FIG. 2 is an exploded view of a microneedle; and

FIGS. 3A and 3B are views of a microneedle before and after dissolution of the plug and exterior wall.

### DETAILED DESCRIPTION

Throughout this disclosure, singular forms such as “a,” “an,” and “the” are used for convenience; however, it should be understood that the singular forms are meant to include the plural unless the singular alone is explicitly specified or is clearly indicated by the context.

Terms such as “common,” “commonly,” “usual,” “usually,” “typical,” and “typically” are used in this disclosure to refer to features that are frequently employed in the disclosure. Unless specifically used in relation to the prior art, these terms are not intended to convey that a feature is present in the prior art. much less that such feature is common, typical, or usual in the prior art.

Use of transdermal devices, and particularly transdermal devices with microneedles, for detecting biological analytes is disclosed, for example, in PCT Publications WO2013/058879, WO2015/008018 and WO2013/58879; EP Patent No. 2898826, U.S. Pat. No. 8,560,059, and U.S. Patent Application Publication No. 2014/0275897. The Applicants have identified a problem with these devices. specifically, that the microneedles impact a biological substrate, typically the skin, at such high rates of speed and under such forces or pressures that even if the microneedles survive the impact without damage the probes contained within the microneedles are often damaged. For example, WO2013/058879 discloses the results of analysis from microneedle-mounted probes on chemical array strips and also discloses that hollow microneedles can survive impact and penetration of biological surfaces such as skin, but does not indicate that the probes can survive the impact and penetration without damage. Thus, the devices as disclosed in the prior art can be, in many cases, inoperable in a practical sense. This is not necessarily because the underlying technology is fundamentally flawed but rather because the probes cannot be delivered into a biological substrate without being damaged to the point where their utility in detecting analyte is compromised.

A solution to this problem, and other problems associated with the use of damageable materials or structures within, or to some extent on, microneedles is disclosed herein. Briefly, a probe for detecting biological materials is located within an exterior wall of a hollow microneedle. The hollow microneedle, which has an opening in the tip, is fitted with a plug covering the tip. The plug is disposed between the probe and the exterior of the hollow microneedle, thus providing a barrier between the probe and the biological surface, which is usually the skin but could be other biological surfaces, that the microneedle impacts and protecting the probe from damage during impaction of the microneedle on the biological surface. The plug is configured to at least partially dissolve in a biological environment to form an opening in the tip of the hollow interior and expose the probe to the biological environment, after which the probe is available to interact with the biological environment and detect an analyte. The plug can be in a variety of forms, and made up of a variety of materials, as further discussed herein.

Any suitable microneedle can be employed. Typically, the microneedle is hollow, and more typically an array of one or more hollow needles is employed. The array of one or more hollow microneedles comprise microneedles having a protruded needle structure that includes one or more exterior walls, the one or more exterior walls defining the sides of a hollow interior of the microneedle.

Microneedles having a generally conical shape are usually employed, in which situations there will typically be only one exterior wall. Other shapes are also possible. For example, some hollow microneedles may be generally conical in shape but have a distinct base portion and end portion each having a different angle with respect to a longitudinal axis of the microneedle. Microneedles can also have generally pyramidal shapes, including without limitation triangular pyramidals and tetrahedral pyramidals, in which case more than one exterior wall will be employed.

The microneedles have a tip configured to face a biological surface that is exterior to the hollow microneedle. The tip will typically have one or more openings at the end or point of the microneedle, however the one or more openings can also be on one or more of the exterior walls or on other portions of the tip. The location of the opening in the microneedle is not particularly relevant to the solution described herein; instead, it is likely to be based on the needs of the particular probe being used, the analyte that is to be detected, and the biological surface that is to be impacted by the microneedles.

The microneedles will typically be in the form of an array of one or more microneedles that protrude from a patch. The patch is usually an adhesive patch comprising an adhesive, such as a pressure sensitive adhesive, to enable the patch and microneedles to adhere to a biological surface while in use, however this is not necessary in all cases because the patch can also be fixed to the biological surface while in use by the operator holding it in place, by medical tape, or by some other means.

Microneedle arrays usable with this disclosure can have a variety of configurations and features, such as those described in the following patents and patent applications, the disclosures of which are incorporated herein by reference. Exemplary microneedle arrays include the structures disclosed in U.S. Patent Application Publication No. 2005/0261631, which describes microneedles having a truncated tapered shape and a controlled aspect ratio. Other exemplary microneedle arrays include the structures disclosed in U.S. Pat. No. 6,091,975, which describes blade-like microprotrusions for piercing the skin. Still other exemplary microneedle arrays include the structures disclosed in U.S. Pat. No. 6,312,612. which describes tapered structures having a hollow central channel. Yet still other exemplary the microneedle arrays include the structures disclosed in U.S. Pat. No. 6,379,324, which describes hollow microneedles having at least one longitudinal blade at the top surface of the tip of the microneedle. A further example of microneedle arrays include the structures disclosed in U.S. Patent Application Publication Nos. 2012/0123387 and 2011/0213335, both of which describe hollow microneedles. Still further exemplary microneedle arrays include the structures disclosed in U.S. Pat. Nos. 6,558,361 and 7,648,484, both of which describe hollow microneedle arrays and methods of manufacturing thereof.

Examples of microneedles that can be employed, either singly or as part of microneedle arrays as disclosed herein, are described in WO2012/074576, which describes liquid crystalline polymer (LCP) microneedles as well as WO2012/122162, which describes a variety of different types and compositions of microneedles that can be employed in the microneedles of the present disclosure.

One or more of the microneedles can be made of a material that is, or that comprises, silicon, glass, or a metal such as stainless steel, titanium, or nickel titanium alloy. A polymeric material, such as a medical grade polymeric material, can also be a component, or the sole component, of one or more of the microneedles. Exemplary types of medical grade polymeric materials include polycarbonate, liquid crystalline polymer (LCP), polyether ether ketone (PEEK), cyclic olefin copolymer (COC), polybutylene terephthalate (PBT). The most commonly used medical grade polymeric materials are polycarbonate and LCP.

One or more of the microneedles can be made of a material that is, or that comprises, a biodegradable polymeric material, particularly, a medical grade biodegradable polymeric material. Exemplary types of medical grade biodegradable materials include polylactic acid (PLA), polyglycolic acid (PGA), PGA and PLA copolymer, polyester-amide polymer (PEA).

The dissolvable plug, or, in cases when the exterior wall of the microneedle is dissolvable, the exterior wall, can be a prepared from a dissolvable, degradable, or disintegradable in a biological environment. A dissolvable, degradable, or disintegradable material is any solid material that dissolves, degrades, or disintegrates during use, typically one that dissolves, degrades, or disintegrates in the tissue underlying the stratum corneum. The dissolvable material can be selected from a carbohydrate or a sugar. In other cases, the dissolvable material is polyvinyl pyrrolidone (PVP). In other cases, the dissolvable material is selected from the group consisting of hyaluronic acid, carboxy methylcellulose, hydroxypropylmethylcellulose, methylcellulose, polyvinyl alcohol, sucrose, glucose, dextran, trehalose, maltodextrin, and a combination thereof.

A microneedle or the plurality of microneedles in a microneedle array useful for practicing the present disclosure can have a variety of shapes that are capable of piercing the stratum corneum. Examples include, square pyramidal shape, triangular pyramidal shape, stepped pyramidal shape, conical shape, microblade shape, or the shape of a hypodermic needle. Commonly, one or more of the plurality of microneedles can have a square pyramidal shape. Commonly, one or more of the plurality of microneedles can have a triangular pyramidal shape. Commonly, one or more of the plurality of microneedles can have a stepped pyramidal shape. Commonly, one or more of the plurality of microneedles can have a conical shape. Commonly, one or more of the plurality of microneedles can have a microblade shape. Commonly, one or mom of the plurality of microneedles can have the shape of a hypodermic needle. The shape can be symmetric or asymmetric. The shape can be truncated (for example, the plurality of microneedles can have a truncated pyramid shape or truncated cone shape).

Often, one or more of the plurality of hollow microneedles in a hollow microneedle array has a conical shape. Commonly, one or more of the plurality of hollow microneedles in a hollow microneedle array can have a cylindrical shape. Commonly, one or more of the plurality of hollow microneedles in a hollow microneedle array can have a square pyramidal shape. Commonly, one or more of the plurality of hollow microneedles in a hollow microneedle army can have a triangular pyramidal shape. Often, one or more of the plurality of hollow microneedles in a hollow microneedle array can have the shape of a hypodermic needle. Particularly, the plurality of hollow microneedles in a hollow microneedle army each have the shape of a conventional hypodermic needle.

In a microneedle array, a plurality of microneedles is typically positioned on a microneedle substrate. Each microneedle has a height h, which is the length from the tip of the microneedle to the microneedle base where the microneedle contacts the substrate. Either the height of a single microneedle or the average height of all microneedles on the microneedle array can be referred to as the height of the microneedle, h. In some cases, each of the plurality of microneedles (or the average of all of the microneedles in the plurality of microneedles) has a height of about 100 to about 3000 micrometers, in some embodiments, about 100 to about 1500 micrometers, in some embodiments, about 100 to about 1200 micrometers, and, in some embodiments, about 100 to about 1000 micrometers. In some cases, each of the plurality of microneedles (or the average of all of the plurality of microneedles) has a height of about 200 to about 1200 micrometers, about 200 to about 1000 micrometers, about 200 to about 750 micrometers, or about 200 to about 600 micrometers.

As an example, each of the plurality of microneedles (or the average of all of the plurality of microneedles) can have a height of about 250 to about 1500 micrometers, about 500 to about 1000 micrometers, or about 500 to about 750 micrometers. As another example, each of the plurality of microneedles (or the average of all of the plurality of microneedles) can have height of about 800 to about 1400 micrometers. Particular values for the height of each of the plurality of microneedles, or the average of all of the plurality of microneedles, in each case in units of micrometers are 3000 or less, 1500 or less, 1200 or less, 1000 or less, 750 or less, or even 600 or less. Other particular values for the height of each of the plurality of microneedles, or the average of all of the plurality of microneedles, in each case in units of micrometers are about 100 or more, about 200 or more, 250 or more, 500 or more, 600 or more, 700 or more 750 or more, or even 800 or more. Particular ranges of values for the height of each of the plurality of microneedles, or the average of all of the plurality of microneedles, in each case in units of micrometers, are 100 to 3000, 250 to 1500, 900 to 1000, and 900 to 950. One particular value for the height of each of the plurality of microneedles, or the average of all of the plurality of microneedles is about 500 micrometers; another particular value for the height of each of the plurality of microneedles, or the average of all of the plurality of microneedles is about 900 micrometers.

A single microneedle or the plurality of microneedles in a microneedle array can also be characterized by their aspect ratio. The aspect ratio of a microneedle is the ratio of the height of the microneedle, h to the width (at the base of the microneedle), w. The aspect ratio can be presented as h:w. In most cases, each of the plurality of microneedles (or the average of all the plurality of microneedles) has (have) an aspect ratio in the range of 2:1 to 5:1. Even more particularly, each of the plurality of microneedles (or the average of all of the microneedles in the plurality of microneedles) has (have) an aspect ratio of at least 3:1.

Any suitable density of microneedles, in terms of number of microneedles per area of the array, can be used, subject only to the area of the base of the microneedles that contacts the substrates. Typically, the array of microneedles contains about 3 to about 30 microneedles per cm² of the array of microneedles.

Any suitable number of microneedles may be used in an array. The number of microneedles can be selected based on the requirements of the particular types of sensor or sensor used and the measurements that are to be taken. For example, if the measurement that is to be taken is considered most accurate when taken in triplicate, for example so that the average of the measurements can be used or so that an error can be detected if the three measurements are not close in value, then at least three microneedles will be present on the array. If multiple sensors are to be used and each sensor requires a separate microneedle to house it, then the number of microneedles on the array will be at least equal to the number of sensors used. Most typically, the array of hollow microneedles contains 3 to 30 hollow microneedles per array of hollow microneedles, such as 3-20 hollow microneedles, 13-20 hollow microneedles, 8-18 hollow microneedles, or even 10 to 30 hollow microneedles per array of hollow microneedles. Particular arrays can have 18 hollow microneedles per army. Other particular arrays can have 12 hollow microneedles per array.

The depth at which the microneedles penetrate the skin is not necessarily the same as the height of the microneedles, because it is possible that the base of the microneedle as well as some of the microneedle near the base does not penetrate the skin. So long as sufficient to deliver the probe to a location where it can perform its intended function, the microneedle is usable with this disclosure. In some cases, each of the plurality of microneedles (or the average of all of the microneedles in the plurality of microneedles) in a microneedle array can penetrate into the skin to a depth of about 50 to about 1500 micrometers, about 50 to about 400 micrometers, or about 50 to about 250 micrometers. In some cases, each of the plurality of microneedles (or the average of all of the microneedles in the plurality of microneedles) in a microneedle array can penetrate into the skin to a depth of about 100 to about 400 micrometers, or about 100 to about 300 micrometers. In some cases, each of the plurality of microneedles (or the average of all of the plurality of microneedles) in a microneedle array can penetrate into the skin to a depth of about 150 to about 1500 micrometers, or about 800 to about 1500 micrometers. In some cases, each of the plurality of microneedles (or the average of all of the microneedles in the plurality of microneedles) in a microneedle array can penetrate into the skin to a depth of about 400 to about 800 micrometers.

Typically, the microneedles are part of a microneedle array and the microneedle array is in turn part of a in the form of a patch, which can include the microneedle array, a skin-contact adhesive, many of which are known in the art, and optionally a backing. Whether on a patch or not, the microneedles can be arranged in any desired pattern or arrangement. For example, the microneedles can be arranged in uniformly spaced rows, which can be aligned or offset. In some cases, the microneedles can be arranged in a polygonal pattern such as a triangle, square, rectangle, pentagon, hexagon, heptagon, octagon, or trapezoid. In other cases, the microneedles can be arranged in a circular or oval pattern. The arrangement of the microneedles will depend on the probe used and what is to be affected or monitored by the probe. The surface area of the patch, which is covered in part, with microneedles will depend on the number of microneedles required, their spacing and arrangement on the patch, and the portion of the body to which the patch is to be delivered. In typical cases, the surface area of the patch can be about 0.1 cm² to about 20 cm², such as about 0.5 cm² to about 5 cm², about 1 cm² to about 3 cm², or even about 1 cm² to about 2 cm².

In some cases, the microneedles can be disposed over substantially the entire surface of the array. This is not required, because it is also possible that a portion of the array or patch is not provided with microneedles. In that case, the portion of the array or patch that does not have microneedles has an area of more than about 1 percent and less than about 75 percent of the total area of the array or patch surface that is designed to be skin-facing (that is, the portion having microneedles protruding therefrom). Thus, given the typical patch areas discussed above, the portion of the patch surface without microneedles typically has an area of more than about 0.65 cm² (0.10 square inch) to less than about 6.5 cm² (1 square inch).

Typically, a hollow channel or bore extends through the microneedle, the channel being surrounded by the exterior wall. In one configuration, the bore exits at a channel opening at or near the tip of the hollow microneedle. The channel usually exits at an opening near the tip of the hollow microneedle. Most commonly, the channel or bore continues along a central axis of the microneedle, but exits like a hypodermic needle on a sloping side-wall of the microneedle to help prevent blockage of the channel by tissue upon insertion. In some cases, the diameter of the channel bore is about 10 to about 200 micrometers. In other cases, the diameter of the channel bore is about 10 to about 150 micrometers. In still other cases, the diameter of the channel bore is about 30 to about 60 micrometers.

The term hollow microneedle also encompasses microneedles that have a hollow trough or divot defined at least in part by the exterior wall of the microneedle. Typically, the trough or divot will run the entire height h of the microneedle, from base to tip, such that a probe or wire can be located within the divot or trough.

The average cross-sectional area of the channel bore is typically about 75 to about 32.000 micrometers, for example about 75 to about 18,000 micrometers, or even about 700 to about 3,000 micrometers.

The microneedle arrays can be defined by the average spacing between adjacent microneedles, and is measured from microneedle tip to adjacent microneedle tip. Typically, the average spacing between adjacent microneedles is about 0.7 mm to about 20 mm, such as about 0.7 mm to about 10 mm, about 2 mm to about 20 mm, or even about 2 mm to about 10 mm. In many cases, the average spacing between adjacent microneedles is greater than about 0.7 mm, such as greater than about 2 mm. In such cases, the average spacing between adjacent microneedles is typically less than about 20 mm, such as less than about 10 mm. As an example, the average spacing from microneedle tip to microneedle. One particular average spacing between adjacent microneedles is about 2 mm. Another particular average spacing between adjacent microneedles is about 0.7 mm.

The microneedle arrays can be manufactured in any suitable way such as by injection molding, compression molding, metal injection molding, stamping, photolithography, or extrusion. In one embodiment, hollow microneedle arrays can be made by thermocycled injection molding of a polymer such as medical grade polycarbonate or LCP, followed by laser drilling to form the channels of the microneedles.

Nonlimiting examples of molding processes for molding polymeric materials into solid microneedle articles can be found in U.S. Pat. No. 8,088,321, U.S. Patent Application Publication Nos. 2012/0258284, and 2012/0041337. A non-limiting example of a publication that discloses the formation of hollow channels in articles comprising microneedles is U.S. Patent Application Publication No. 2015/0306363, which is incorporated herein by reference in its entirety.

Microneedle-based probes for detecting biological analytes have been described, for example, in PCT Publications WO2013/058879. WO2015/008018 and WO2013/58879; EP Patent No. 2898826. U.S. Pat. No. 8,560,059, and U.S. Patent Application Publication No. 2014/0275897. Any type of probe for detecting analyte can be employed. The nature of the probe will depend on the analyte that is to be detected. Each hollow microneedle may contain one or more probes.

Any type of probe that is small enough to fit inside the exterior wall of a hollow microneedle can be used. Exemplary probes include those designed for lactate measuring and described in EP2898826, which can be situated inside microneedles as described in that document.

Another exemplary probe comprises an array of filaments, wherein each filament comprises a substrate and a conductive layer coupled to the substrate and configured to facilitate analyte detection. Each filament can comprise an insulating layer configured to isolate regions defined by the conductive layer for analyte detection, a sensing layer coupled to the conductive layer, the sensing layer configured to enable transduction of an ionic concentration into an electronic voltage, and a selective coating coupled to the sensing layer configured to facilitate detection of specific target analytes or ions. Such probes, as well as methods of attaching them to microneedles, are described in U.S. Patent Application Publication No. 2014/0275897.

Yet other exemplary probes include nanowires fixed to the interior surface of a hollow microneedle. Such nanowires can have a membrane covering, and can be configured to detect glucose as further described in WO2015/008018.

Optical sensing probes, such as those disclosed in U.S. Pat. No. 8,560,059, can also be employed within hollow microneedles.

The one or more probes as described in WO2013/58879 are most often employed. Briefly, such probes can be coated with a functionalized coating configured to interact with an analyte, particularly an analyte in a fluid such as a biological fluid. The functionalized coating can be configured, for example, to immobilize an analyte at an interface of the coating and the fluid by electropolymerization, polymer entrapment, electrostatic interaction, covalent attachment, or adsorption. Typical functional coatings are enzyme-functionalized coatings and ion-selective coatings. Once immobilized or otherwise interacting with the probe, typically with the functional coating, the analyte can have an electrochemical interaction with the functionalized coating. The one or more probes can be configured to detect the electrochemical interaction, for example by way of amperometry, voltammetry, or potentiometry.

As described in detail in WO2013/58879, the probe can be a component of an electrode. Depending on the analyte to be detected, the probe can be made of any suitable material. Typical materials include one or more of carbon fiber, carbon paste, conducting metal, semiconducting polymer, and conducting polymer

The one or more probes, having detected or sensed the analyte, for example by way of the analyte's interaction with the coating, can produce a probe sensing signal. One or more properties of probe sensing signal, such as the type of signal, the magnitude of the signal, etc. The probe sensing signal is typically an electrical signal, but can also be electromagnetic radiation such as luminescence. When the probe sensing signal is not an electronic signal, it is usually converted into an electronic signal, such as by way of a charge coupled device or other device known in the art, for ease of use, however this is not required.

Most commonly only one probe is contained within each hollow microneedle. It is also possible that, in an array of microneedles, different probes can be contained within different microneedles. For example, half of the microneedles in an array can have a lactate sensing probe as described in EP2898826 within the one or more exterior walls, and the other half can have probes with functional coatings for sensing glutamate as disclosed in WO2013/58879.

A processing unit can be in communication with the one or more probes. The processing unit is most often an external processing unit, meaning that it is not integrated with the microneedle array and/or patch; however, it is also possible for the processing unit to be on the array, in which case it is typically located on the side of the adhesive patch that does not have the hollow microneedles. The processing unit can be configured to receive the probe sensing signals, which are usually in the form of electronic signals. The processing unit can be configured to analyze the probe sensing signals. For example, the processing unit can be configured to compare the probe sensing signals to one or more threshold signals to determine wither the analyte condition reflects a healthy state or a diseased state. As another example, the processing unit can be configured to determine a pattern in the probe sensing signals, particularly when the analyte is detected on multiple occasions or continuously over the course of a time period, and indicate whether the analyte concentration indicates a healthy state or a diseased state. As another example, the processing unit can be configured to multiplex the probe sensing signal.

To facilitate the processing unit being in communication with the one or more probes, the device can further comprise one or more wires coupled to the probes, each wire being electrically conductive and configured to transmit the probe sensing signal produced by the one or more probes. As an alternative to wires, the device can employ one or more wireless communication modules to transmit the probe sensing signal produced by the one or more probes to the processing unit.

The plug covers a tip of the hollow needle such that the plug is disposed between the probe and the exterior of the hollow microneedle. In this manner, the plug protects the probe from bearing the full force of the impact of the microneedle on the biological surface, such as the skin. Regardless of the configuration, the plug at least partially dissolves in a biological environment to form an opening in the top of the hollow interior, thereby exposing the probe to the biological environment.

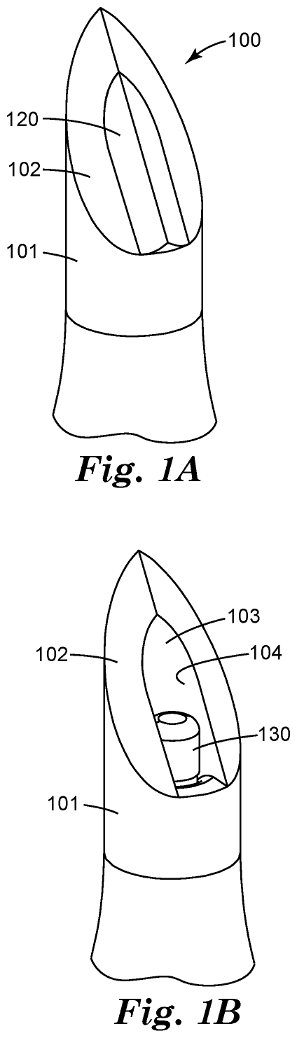

The plug can be configured in a variety of ways. For example, it can be configured so that at least a portion of the plug is disposed within the hollow interior of the microneedle. An example of this configuration is shown in FIG. 1A microneedle 100 comprises exterior wall 101 and tip 102. Plug 120 is present in tip 102. FIG. 1B shows the same microneedle 100 after exposure to a biological environment has caused plug 120 to dissolve exposing opening 103 in tip 102 as well as the hollow interior 104 of microneedle 100. Located inside the hollow interior 104 of microneedle 100 is probe 130, which, in FIG. 1B, is exposed to an exterior environment where it can sense analyte.

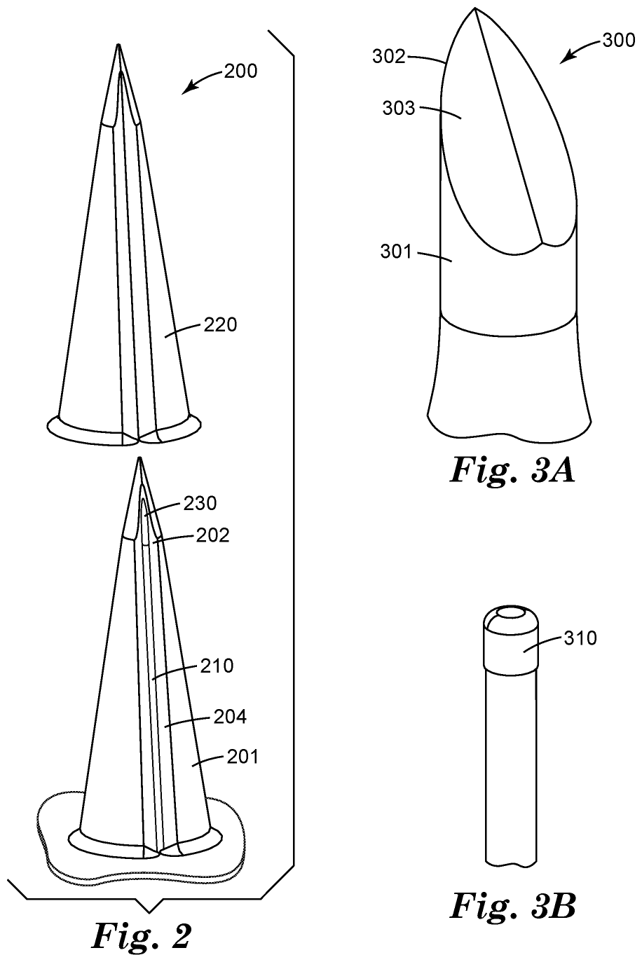

The plug can also be configured as a cap that covers at least the tip of the microneedle or a sheath that covers both the tip of the microneedle and at least a portion of the exterior wall of the microneedle. An example of the latter is shown in FIG. 2, which is an exploded view of microneedle 200. Microneedle 200 features exterior wall 201 and tip opening 202. Within hollow interior 204 of microneedle 200 which in this FIG. 2 is a divot running the length of microneedle 200, is probe 210. Plug 220, which is configured like a sheath, is being placed over microneedle 200 to cover tip opening 202 and protect probe 230 from being damage upon impact with a biological surface.

Regardless of how it is disposed on or within the hollow microneedle, the plug is configured to at least partially dissolve in a biological environment. Typically, the plug is made in whole or in part out of a dissolvable material that dissolves upon contact with a biological fluid, such as interstitial fluid, blood, water, or the like. Any dissolvable material that does not have unduly harmful effects on the subject can be used. Typical materials are sugars, natural polymers, or synthetic polymers. When a sugar is used, it is typically fructose, trehalose, raffinose, sucrose, or glucose. When a natural polymer is employed, it is typically cellulose or sodium alginate. When a synthetic polymer is employed, it is typically carboxymethylcellulose, polyvinylpyrolidone, poly(ethylene glycol), poly(ethylene oxide), poly(vinyl alcohol), polylactide, polyglycolide, polycapralactone, or a copolymer of two or more of lactide. glycolide, and capralactone.

The plug can be produced by any suitable means. The means of producing the plug will depend upon the configuration of the plug and the material that it is made out of. For example, a plug that is in the form of a cap on top of the tip of the microneedle or a sheath over the tip and a portion of the exterior wall of the microneedle can be made by way of conventional coating, such as dip-coating, spray-coating, or the like, using a solution or dispersion of the constituents of the plug. When the plug is disposed only within an opening in the hollow microneedle, then it can be made by a process analogous to dip-coating wherein the tip of the hollow microneedle is contacted with a solution or dispersion of the material that constitutes the plug so as to draw up the solution or dispersion into the opening in the tip of the microneedle by capillary action. Subsequent drying can leave the plug in place. Alternatively, the plug may be manufactured separately from the microneedle and then inserted into or onto the microneedle by conventional manufacturing methods.

Most commonly, the exterior wall is not dissolvable, degradable, or disintegrable in a biological environment.

However, it is possible for all or part of the exterior wall of the microneedle to be made from a dissolvable material, which can be any dissolvable material and particularly those mentioned herein with respect to the plug. For example, the exterior wall can be made up of a mesh support of non-dissolvable material, such as metal or a non-dissolvable plastic, with the solid dissolvable material located between or around the mesh support. Alternatively, the entire exterior wall can be made of a dissolvable material. In either case, the exterior wall can either be separate from the plug or integral with the plug.

FIGS. 3A and 3B show an example of the latter case. FIG. 3A shows microneedle 300 featuring exterior wall 301 and tip 302 with plug 303 in the tip. Both exterior wall 301 (including tip 302) and plug 303 are made of dissolvable materials. Upon contact with a biological fluid, exterior wall 301 (including tip 302) and plug 303 dissolve, exposing probe 310 to a biological environment (not shown) where it can function to sense analytes.

The devices as described herein can be used in a method of detecting an analyte. The analyte is typically a biological molecule, such as a biochemical, a metabolite, or an electrolyte. The method can comprise impacting a biological surface with one or microneedles of a device as described herein to pierce the biological surface with the one or more microneedles. After a sufficient time has passed to allow the plug, and dissolvable exterior wall if employed, to dissolve, then the analyte can be detected. Typically, dissolution of the dissolvable materials of the plug or exterior wall occurs in less than 30 minutes, such as less than 15 minutes, less than 10 minutes, less than 5 minutes, less than 3 minutes, less than 1 minute, or even less than 30 seconds after piercing of the biological surface.

The biological surface that is impacted by the microneedles is usually skin, particularly the epidermis or the dermis. However, other biological surfaces such as membranes, such as mucosal membranes, or the surface of the eye, can also be pierced by the microneedles in the method.

The following is a list of exemplary illustrative embodiments of the disclosure. These embodiments are not intended to be limiting, but are provided to assist the artisan in understanding particular ways in which the devices and methods of the disclosure can be implemented.

1. A device comprising:

an array of one or more hollow microneedles, the one or more microneedles comprising
a protruded needle structure including one or more exterior walls that define the sides of a hollow interior,
a tip configured to face a biological surface that is exterior to the hollow microneedle, and
a probe located within the hollow interior defined by the one or more exterior walls, wherein

the probe is configured to interact with one or more chemical or biological substances that come into contact with the probe to produce a probe sensing signal; and

characterized in that the device further comprises a plug covering a tip of the hollow needle such that the plug is disposed between the probe and the exterior of the hollow microneedle, the plug being configured to at least partially dissolve in a biological environment to form an opening in the tip of the hollow interior and expose the probe to the biological environment.

2. The device of embodiment 1, wherein one or more of the probes includes a functionalized coating configured to interact with an analyte within a fluid.

3. The device of embodiment 2, wherein the functionalized coating includes at least one of an enzyme-functionalized coating or an ion-selective coating.

4. The device of any of the preceding embodiments, wherein the analyte is a biochemical, a metabolite, an electrolyte, an ion, a pathogen, a microorganism, or a combination of any of the foregoing.

5. The device of any of any of embodiments 24, wherein at least one of the functionalized coating of the probes is configured to detect an electrochemical interaction between the analyte and the coating on by way of amperometry, voltammetry, or potentiometry.

6. The device of any of embodiments 2-5, wherein the analyte is immobilized at an interface of the coating by at least one of electropolymerization, polymer entrapment, electrostatic interaction, covalent attachment, or adsorption.

7. The device of any of embodiments 2-6, further comprising a processing unit in communication with the probe to receive the probe sensing signals.

8. The device of embodiment 7, wherein the processing unit is configured to compare the probe sensing signals to one or more threshold values to determine whether the analyte condition reflects a healthy state or a diseased state.

9. The device of any of embodiments 7-8, wherein the processing unit is configured to determine a pattern in the probe sensing signals that indicates whether the analyte concentration reflects a healthy or diseased state.

10. The device of any of embodiments 7-9, wherein the processing unit is configured to multiplex the probe sensing signals.

11. The device of any of the preceding embodiments, wherein the array of one or more hollow microneedles is attached to an adhesive patch.

12. The device of any of the preceding embodiments, wherein the device further comprises one or more wires coupled to the probes, each wire being electrically conductive and configured to transmit the probe sensing signal produced by one or more probes.

13. The device of any of the preceding embodiments, wherein the device further comprises at least one wireless communication module that is configured to transmit the probe sensing signal produced by one or more robes.

14. The device of any of the preceding embodiments, wherein the plug comprises a sugar, a natural polymer, or a synthetic polymer.

15. The device of embodiment 14, wherein the sugar is fructose, trehalose, raffinose, sucrose, or glucose.

16. The device of embodiment 14, wherein the natural polymer is cellulose or sodium alginate.

17. The device of embodiment 14, wherein the synthetic polymer is carboxymethylcellulose, polyvinylpyrrolidone, poly(ethylene glycol), poly(ethylene oxide), poly(vinyl alcohol), polylactide, polyglycolide, polycapralactone, or a copolymer of two or more of lactide, glycolide, and capralactone.

18. The device of any of the preceding embodiments, wherein at least a portion of the plug is disposed within the hollow interior of the hollow microneedles.

19. The device of any of the proceeding embodiments, wherein the plug is disposed as a cap covering the tip of the hollow microneedle.

20. The device of any of the preceding embodiments, wherein the plug is disposed as a sheath covering the tip of the hollow microneedle and at least a portion of the exterior wall of the microneedle.

21. The device of any of the preceding embodiments, wherein at least a portion of the exterior wall is formed out of a dissolvable material that is configured to at least partially dissolve in a biological environment to leave one or more opening in the exterior wall.

22. The device of embodiment 21, wherein the dissolvable material comprises a sugar, a natural polymer, or a synthetic polymer.

23. The device of embodiment 21, wherein the sugar is fructose, trehalose, raffinose, sucrose, or glucose.

24. The device of embodiment 21, wherein the natural polymer is cellulose or sodium alginate.

25. The device of embodiment 21, wherein the synthetic polymer is carboxymethylcellulose, polyvinylpyrrolidone, poly(ethylene glycol), poly(ethylene oxide), poly(vinyl alcohol), polylactide, polyglycolide, polycapralactone, or a copolymer of two or more of lactide, glycolide, and capralactone.

26. The device of any of the preceding embodiments, further comprising an electrode, the electrode comprising the probe.

27. The device of any of the preceding embodiments, wherein the probe signal is an electrical signal.

28. The device of any of the preceding embodiments, wherein the probe comprises one or more of carbon fiber, carbon paste, conducting metal, semiconducting polymer, and conducting polymer.

29. The device of any of the preceding embodiments, wherein the probe comprises at least one of an enzyme-functionalized coating or an ion-selective coating.

30. The device of any of embodiments 26-29, wherein the electrode comprises a functionalized coating for interacting with an analyte

31. The device of embodiment 30, wherein the functionalized coating comprises at least one of an enzyme-functionalized coating or an ion-selective coating.

32. The device of any of embodiments 26-31, wherein the analyte interacts with the electrode or with a functionalized coating on the electrode, and wherein the probe is configured to detect interaction of the electrode or the functionalized coating with the analyte is by at least one of amperometry, potentiometry, or voltammetry.

33. A method of detecting an analyte, the method comprising

piercing a biological substrate with the hollow microneedles of the device of any of the preceding embodiments;

allowing the analyte to come into operative proximity with the probe to produce a probe signal that correlates to the presence of the analyte.

34. The method of embodiment 33, wherein the probe signal corresponds to the concentration of the analyte.

35. The method of any of embodiments 33 or 34, wherein the biological substrate comprises an epidermis.

36. The method of any of embodiments 33 or 34, wherein the biological substrate comprises a dermis.

37. The method of any of embodiments 33 or 34, wherein the biological substrate comprises a mucosal membrane.

1. A device comprising:
an array of one or more hollow microneedles, the one or more microneedles comprising
a protruded needle structure including one or more exterior walls that define the sides of a hollow interior,
a tip configured to face a biological surface that is exterior to the hollow microneedle, and
a probe located within the hollow interior; wherein
the probe is configured to interact with one or more chemical or biological substances that come into contact with the probe to produce a probe sensing signal; and
characterized in that the device further comprises a plug covering a tip of the hollow needle such that the plug is disposed between the probe and the exterior of the hollow microneedle, the plug being configured to at least partially dissolve in a biological environment to form an opening in the tip of the hollow interior and expose the probe to the biological environment.

2. The device of claim 1, wherein at least a portion of the exterior wall is formed out of a material that is configured not to dissolve, disintegrate, or degrade in a biological environment.

3. The device of claim 1, wherein one or more of the probes includes a functionalized coating configured to interact with an analyte within a fluid.

4. The device of claim 1, wherein the functionalized coating includes at least one of an enzyme-functionalized coating or an ion-selective coating.

5. The device of claim 1, wherein the array of one or more hollow microneedles is attached to an adhesive patch.

6. The device of claim 1, wherein the plug comprises a sugar, a natural polymer, or a synthetic polymer.

7. The device of claim 6, wherein the sugar is fructose, trehalose, raffinose, sucrose, or glucose.

8. The device of claim 6, wherein the natural polymer is cellulose or sodium alginate.

9. The device of claim 6, wherein the synthetic polymer is carboxymethylcellulose, polyvinylpyrrolidone, poly(ethylene glycol), poly(ethylene oxide), poly(vinyl alcohol), polylactide, polyglycolide, polycapralactone, or a copolymer of two or more of lactide, glycolide, and capralactone.

10. The device of claim 1, wherein at least a portion of the plug is disposed within the hollow interior of the microneedle.

11. The device of claim 1, wherein the plug is disposed as a cap covering the tip of the hollow microneedle.

12. The device of claim 1, wherein the plug is disposed as a sheath covering the tip of the hollow microneedle and at least a portion of the exterior wall of the microneedle.

13. The device of claim 1, further comprising an electrode, the electrode comprising the probe.

14. The device of claim 1, wherein the probe signal is an electrical signal.

15. The device of claim 1, wherein the probe comprises one or more of carbon fiber, carbon paste, conducting metal, semiconducting polymer, and conducting polymer.

16. The device of claim 1, wherein the device further comprises one or more wires or one or more wireless communication modules coupled to the probes, the wires or wireless communication modules being configured to transmit the probe sensing signal produced by one or more probes.

17. A method of detecting an analyte on a biological substrate, the method comprising
piercing the biological substrate with the hollow microneedles of the device of claim 1;
allowing the analyte to come into operative proximity with the probe to produce a probe signal that correlates to the presence of the analyte.

18. The method of claim 17, wherein the biological substrate comprises an epidermis.

19. The method of claim 17, wherein the biological substrate comprises a dermis.

20. The method of claim 17, wherein the biological substrate comprises a mucosal membrane.
